(19) United States
(12) Patent Application Publication
(10) Pub. No.: US 2009/0118704 A1
(43) Pub. Date: May 7, 2009

(54) INTERCONNECTED RIBBON COILS, MEDICAL DEVICES INCLUDING AN INTERCONNECTED RIBBON COIL, AND METHODS FOR MANUFACTURING AN INTERCONNECTED RIBBON COIL

(75) Inventors: James S. Sharrow, Bloomington, MN (US); Brian R. Reynolds, Ramsey, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., MAPLE GROVE, MN (US)

(21) Appl. No.: 11/934,682

(22) Filed: Nov. 2, 2007

(51) Int. Cl. A61M 25/00

(52) U.S. Cl. 604/523; 29/605

(57) ABSTRACT

Medical devices and methods for making and using the same. An example method for manufacturing a medical device, or one or more components thereof, may include providing a tubular member and laser cutting the tubular member to define an interconnected ribbon coil. An example medical device may include a core member and an interconnected ribbon coil disposed about a portion of the core member.

### FIELD OF THE INVENTION

The present invention pertains to medical devices, and methods for manufacturing medical devices. More particularly, the present invention pertains to interconnected ribbon coils, methods for manufacturing an interconnected ribbon coil, and methods for manufacturing a medical device with an interconnected ribbon coil.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices or components thereof. An example method for manufacturing a medical device, or one or more components thereof, may include providing a tubular member and laser cutting the tubular member to define an interconnected ribbon coil. An example medical device may include a core member and an interconnected ribbon coil disposed about a portion of the core member.

The above summary of some embodiments is not intended to describe each disclosed embodiment or every implementation of the present invention. The Figures, and Detailed Description, which follow, more particularly exemplify these embodiments

### BRIEF DESCRIPTION OF THE DRAWINGS

The invention may be more completely understood in consideration of the following detailed description of various embodiments of the invention in connection with the accompanying drawings, in which:

FIG. 1 is a plan view of an example medical device disposed in a blood vessel;

FIG. 2 is a partial cross-sectional view of an example medical device; and

FIGS. 3-10 are perspective views of example tubular members or portions of example tubular members. These figures also illustrate some of the example interconnected ribbon coils that may be formed from the tubular members.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

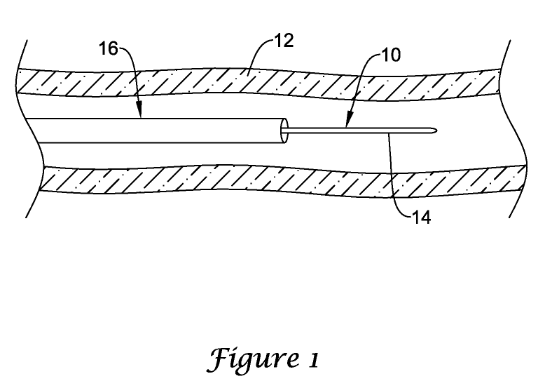

FIG. 1 is a plan view of an example medical device 10, for example a guidewire, disposed in a blood vessel 12. Guidewire 10 may include a distal section 14 that may be generally configured for probing within the anatomy of a patient. Guidewire 10 may be used for intravascular procedures. For example, guidewire 10 may be used in conjunction with another medical device 16, which may take the form of a catheter, to treat and/or diagnose a medical condition. Of course, numerous other uses are known amongst clinicians for guidewires, catheters, and other similarly configured medical devices.

Although medical device 10 is depicted in several of the drawings as a guidewire, it is not intended to be limited to just being a guidewire. Indeed, medical device 10 may take the form of any suitable guiding, diagnosing, or treating device (including catheters, endoscopic instruments, laparoscopic instruments, etc., and the like) and it may be suitable for use at essentially any location and/or body lumen within a patient. For example, medical device/guidewire 10 may be suitable for use in neurological interventions, coronary interventions, peripheral interventions, etc. As such, guidewire 10 may be appropriately sized for any given intervention. For example, guidewire 10 may have an outside diameter of about 0.001 to 0.5 inches or about 0.0015 to 0.05 inches for neurological interventions; an outside diameter of about 0.001 to 0.5 inches or about 0.01 to 0.05 inches for coronary interventions; or an outside diameter of about 0.01 to 0.5 inches or about 0.02 to 0.05 inches for peripheral interventions. These dimensions, of course, may vary depending on, for example, the type of device (e.g., catheter, guidewire, etc.), the anatomy of the patient, and/or the goal of the intervention. In at least some embodiments, for example, guidewire 10 may be a crossing guidewire that can be used to help a clinician cross an occlusion or stenosis in vessel 12.

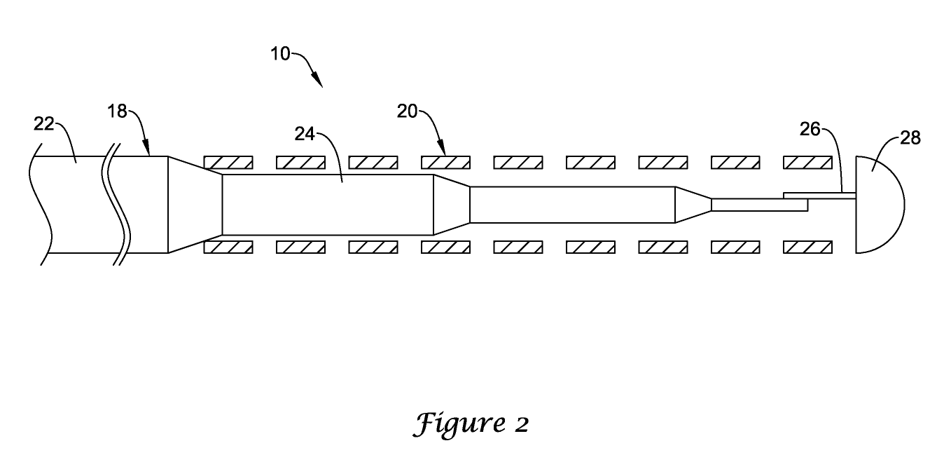

FIG. 2 is a partial cross-sectional view of guidewire 10. Here it can be seen that guidewire 10 may include a core member or core wire 18 and an interconnected ribbon coil 20 disposed over at least a portion of core wire 18. Core wire 18 may include a proximal section 22 and a distal section 24. A connector (not shown) may be disposed between and attach proximal section 22 to distal section 24. Alternatively, core wire 18 may be a unitary member without a connector. A shaping member 26 may be coupled to core wire 18 (for example distal section 24 of core wire 18), interconnected ribbon coil 20, or both. Shaping member 26 may be made from a relatively inelastic material so that a clinician can bend or shape the distal end of guidewire 10 into a shape that may facilitate navigation of guidewire 10 through the anatomy. Some examples of suitable materials for core wire 18, interconnected ribbon coil 20, shaping member 26, etc. can be found below. A tip member 28 may also be coupled to core wire 18, interconnected ribbon coil 20, or both that may define an atraumatic distal tip of guidewire 10. In general, tip member 28 may include solder. However, other versions of tip member 28 are contemplated including tip members 28 that comprise or form a polymeric tip.

As indicated above, guidewire 10 may include interconnected ribbon coil 20. In at least some embodiments, being “interconnected” may be understood to mean that at least some of the individual windings of interconnected ribbon coil 20 are joined together. Interconnecting at least some of the windings of interconnected ribbon coil 20, in at least some embodiments, may be understood to mean that the “coil” is structurally altered so that it may not actually be a “coil” as traditionally understood. Instead, interconnected ribbon coil 20 may be more accurately described as a hybrid tube-coil structure that combines some of the desirable features of a coil with those of a tube without taking the form of either structure. For example, interconnected ribbon coil 20 may combine some of the beneficial flexibility characteristics of a coil with the desirable torque-transmitting characteristics of a tube or slotted tube without actually being a coil or a tube.

Manufacturing interconnected ribbon coil 20 may be achieved in a number of different ways. In at least some embodiments, interconnected ribbon coil 20 may be formed by laser cutting a tube in a manner that defines the desired structure. Several examples of how this might occur as well as variations in form for a number of example ribbon coils are described in more detail below. While laser cutting may be one method that may be utilized for forming interconnected ribbon coil 20, this is not intended to be limiting as other methods are contemplated including micro-machining, saw-cutting (e.g., using a diamond grit embedded semiconductor dicing blade), electron discharge machining, grinding, milling, casting, molding, chemically etching or treating, or other known methods, and the like.

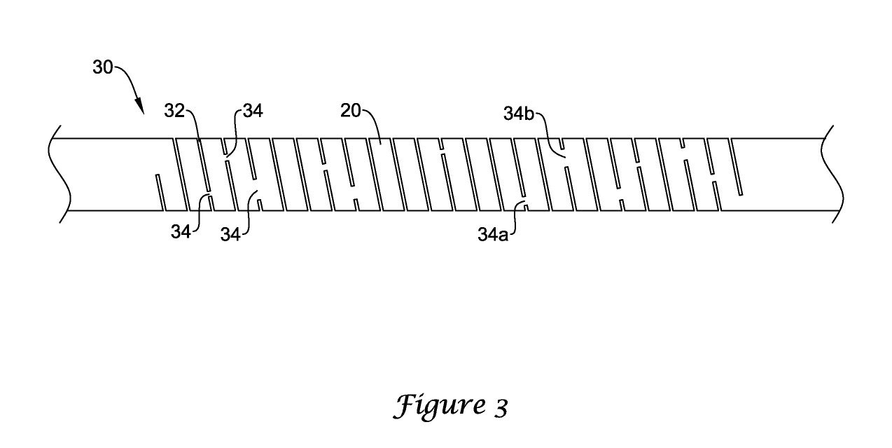

Turning now to FIG. 3, here a tubular member 30 is shown that has a slot 32 extending helically about tubular member 30 to define interconnected ribbon coil 20. Slot 32 may be laser cut into tubular member 30. It should be noted that FIGS. 3-10 bear the same reference numbers for like-named structures. The labeling of such structures is utilized primarily for convenience and is not intended to suggest that all of the structures depicted therein are the same. Indeed, FIG. 3-10 are intended to depict variations in the laser cutting process and the resultant variations in a number of different interconnected ribbon coils 20. It should also be noted that although all of the permutations that can be achieved by mixing and matching the variations depicted in FIGS. 3-10 are not expressly shown, interconnected ribbon coils 20 are contemplated that incorporate essentially all of the suitable combinations of the structural arrangements described below. Furthermore, just because a particular figure may not depict a particular structural arrangement, it should not be assumed that the arrangement depicted in that figure cannot include such a structural arrangement as some of the permutations are intentionally left out for the sake of clarity in describing some of the example arrangements.

It can be seen in FIG. 3 that portions of tubular member 30 may remain “uncut”, thus defining a number of breaks or interruptions in slot 32. Some of these “interruptions” bear reference 34 in FIG. 3 and represent locations along tubular member 30 where the laser cutting process was stopped or skipped. These uncut tube wall region 34 form the interconnections between the individual winding of interconnected ribbon coil 20.

As it can be seen in FIG. 3, several variations in the laser cutting process can be utilized to change the structure of interconnected ribbon coil 20 and, consequently, produce a wide variety of different embodiments of interconnected ribbon coil 20. For example, the position of uncut tube wall regions 34 along the longitudinal axis of interconnected ribbon coil 20 (and/or tubular member 30) can vary. In some embodiments, essentially all of the uncut tube wall regions 34 line up along the longitudinal axis. In other embodiments, adjacent uncut tube wall regions 34 are rotated about the longitudinal axis. For example, adjacent uncut tube wall regions 34 may be rotated a fixed amount (e.g., 30, 45, 60, 75, 90, 105, 120, 135, 150, 165, 180, 195, 210, 225, 240, 255, 270, 285, 300, 315, 330, 345, or more or less degrees, including essentially any suitable number of degrees between 0 and 360), a variable amount, or combinations thereof. In still other embodiments, interconnected ribbon coil 20 may include some uncut tube wall regions 34 that are longitudinally aligned and others that are rotated relative to one another.

Other variations are contemplated including the length of the uncut tube wall regions 34. The “length” of uncut tube wall regions 34 is understood to be the distance along helical slot 32 where adjacent helically cut regions are separated from one another. This might otherwise be described as being the length between where laser cutting slot “stops” and then “starts” again. In some embodiments, the lengths of all the uncut tube wall regions 34 are same. In other embodiments, at least some of the uncut tube wall regions 34 have different lengths. For example, one uncut wall region 34a is depicted in FIG. 3 that can be described as being relatively “short” relative to another 34b. It can be appreciated that a vast number of different embodiments of interconnected ribbon coil 20 are contemplated that utilize different arrangements of uncut tube wall regions 34 and different lengths thereof.

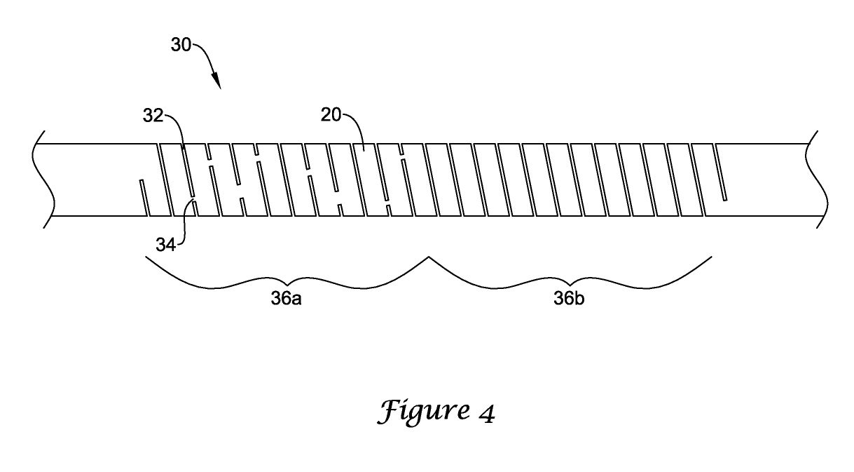

Another variation is shown in FIG. 4. Here, helical slot 32 is arranged such a different number of uncut tube regions 34 are distributed along different portions of interconnected ribbon coil 20. For example, a first portion 36a of interconnected ribbon coil 20 may include relatively more uncut tube regions 34 per unit length than a second portion 36b. The relative location (i.e., proximal, distal, medial, etc.) of portions 36a/36b can vary. For example, in some embodiments, first portion 36a (bearing “more” uncut tube regions 34) is located closer to the proximal end of interconnected ribbon coil 20 whereas second portion 36b may be located more distally. The converse, of course, is also contemplated as well as numerous other variations. Indeed, some embodiments of interconnected ribbon coil 20 are contemplated that include addition portions bearing a different frequency of uncut tube regions 34.

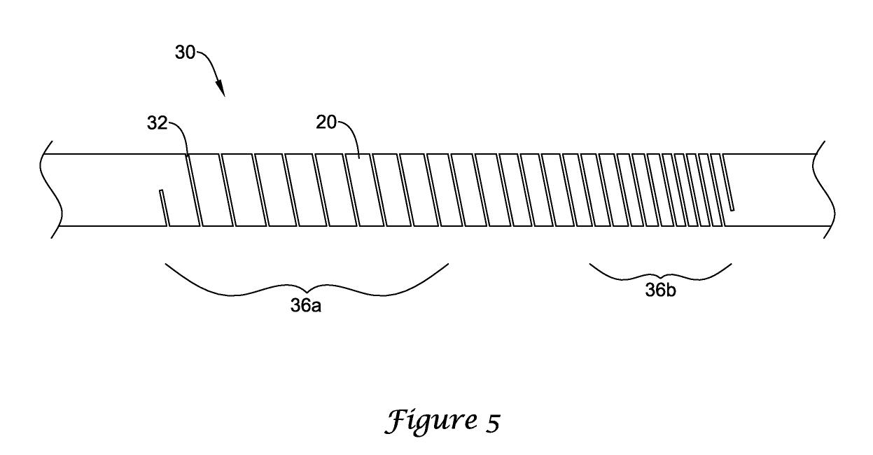

FIG. 5 illustrates another variation. Here, helical slot 32 traces a different pitch such that the “width” of interconnected ribbon coil 20. For example, first portion 36a may include an interconnected ribbon coil 20 with an increased width relative to second portion 36b. In some embodiments, first portion 36a, second portion 36b, or any portions between or apart from portions 36a/36b may include an interconnected ribbon coil 20 with a variable or changing width. For example, the width of interconnected ribbon coil 20 may gradually change from “wide” in first portion 36a to “narrow” in second portion 36 as well as change within portion 36a, 36b, or both.

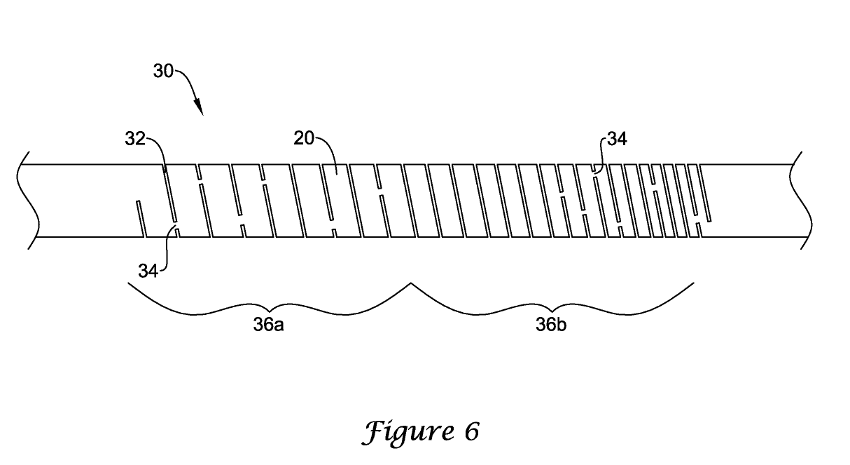

FIG. 6 illustrates a version of tubular member 30/interconnected ribbon coil 20 that combines the features depicted in FIGS. 4 and 5. For example, first portion 36a in FIG. 6 includes a relatively wide interconnected ribbon coil 20 with relatively frequent uncut tube regions 34. Second portion 36b includes a relatively narrow interconnected ribbon coil 20 with relatively infrequent or less frequent uncut tube regions 34. This figure helps to illustrate that the various configurations of interconnected ribbon coils 20 can be combined in mixed in any suitable way.

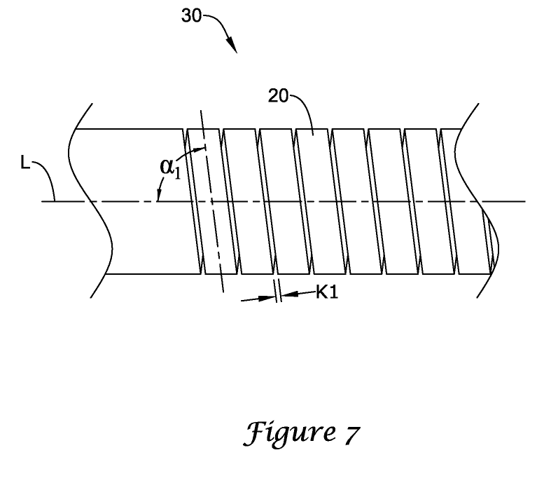

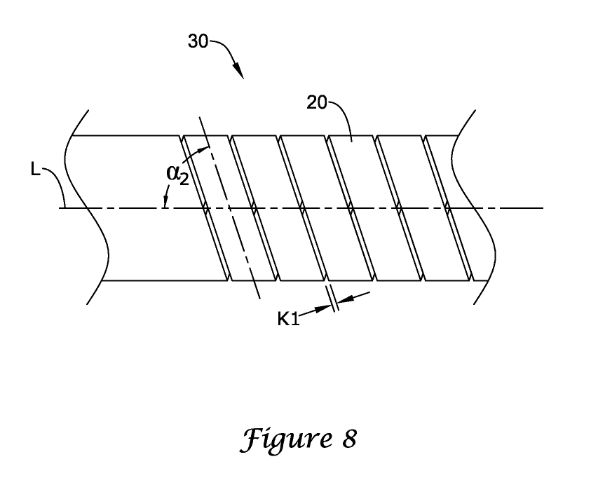

FIGS. 7-10 illustrate additional variations for interconnected ribbon coils 20 that can be included with essentially any suitable embodiment. For example, FIGS. 7 and 8 illustrate interconnected ribbon coils 20 that differ only in the angle that the interconnected ribbon coil 20 is oriented at relative to the longitudinal axis L of tubular member 30. In FIG. 7, the windings of interconnected ribbon coil 20 are oriented at a first angle α₁ relative to the longitudinal axis L whereas the windings of interconnected ribbon coil 20 in FIG. 8 are oriented at a second angle α₂ relative to the longitudinal axis L. In at least some embodiments, both angles α₁/α₂ are different acute angles. However other arrangements are contemplated where one or both of angles α₁/α₂ are obtuse.

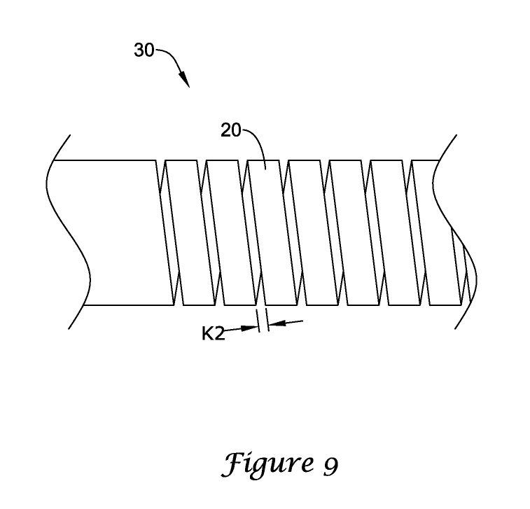

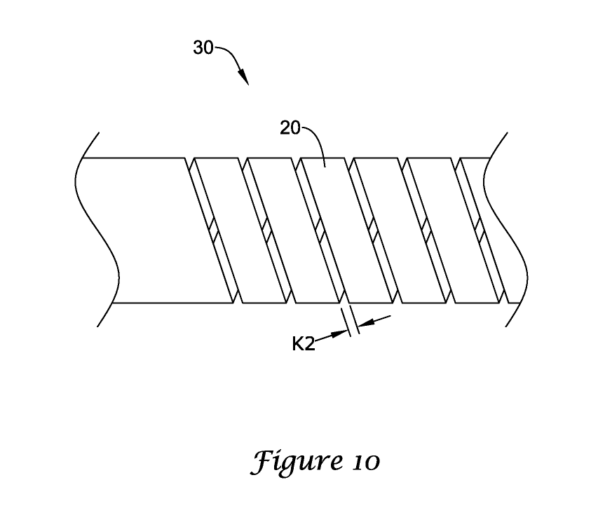

FIGS. 9 and 10 illustrate similar versions of the tubular members 30/interconnected ribbon coils 20 depicted in FIGS. 7 and 8, respectively, that differ in the amount of spacing between individual windings of interconnected ribbon coils 20 or kerf. For example, in FIGS. 7 and 8, the windings of interconnected ribbon coils 20 are spaced so as to have a first kerf K₁. Conversely, in FIGS. 9 and 10, the windings of interconnected ribbon coils 20 are spaced so as to have a second kerf K₂ that is different (in this case larger) than K₁.

With the above discussion in mind, additional variations are contemplated for the various structures described above including difference in materials. For example, the materials that can be used for the various components of guidewire 10 or components or subassemblies thereof such as interconnected ribbon coil 20, tubular member 30, etc. may include those commonly associated with medical devices. For example, any of the structures disclosed herein may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: NO6625 such as INCONEL® 625, UNS: NO6022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of guidewire 10 or any of the subassemblies or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of guidewire 10 to achieve the same result.

In some embodiments, a degree of MRI compatibility is imparted into guidewire 10. For example, to enhance compatibility with Magnetic Resonance Imaging (MRI) machines, it may be desirable to make one or more parts of guidewire 10, in a manner that would impart a degree of MRI compatibility. For example, guidewire 10 or portions thereof may be made of a material that does not substantially distort the image and create substantial artifacts (artifacts are gaps in the image). Certain ferromagnetic materials, for example, may not be suitable because they may create artifacts in an MRI image. Guidewire 10 or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In addition to the metals disclosed above, guidewire 10 and/or components or subassemblies thereof may include a polymer. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP. In some embodiments, a portion of interconnected ribbon coil 20 (e.g., a distal portion) may be embedded within a polymer jacket that is made from any of the polymers listed herein.

In some embodiments, the exterior surface of the guidewire 10 may be sandblasted, beadblasted, sodium bicarbonate-blasted, electropolished, etc. In these as well as in some other embodiments, a coating, for example a lubricious, a hydrophilic, a protective, or other type of coating may be applied over portions or all of guidewire 10. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A method for manufacturing a ribbon coil for a medical device, comprising the steps of:
providing a tubular member, the tubular member including a nickel-titanium alloy;
laser cutting a slot in the tubular member to define an interconnected ribbon coil, wherein the slot follows an interrupted helical pattern about the tubular member and includes a plurality of helically cut regions interspersed with uncut tube wall regions; and
wherein a first region of the interconnected ribbon coil has a first ribbon width and a second region of the interconnected ribbon coil has a second ribbon width different from the first ribbon width.

2. The method of claim 1, wherein the tubular member has a longitudinal axis and wherein adjacent uncut tube wall regions are aligned along the longitudinal axis.

3. The method of claim 1, wherein the tubular member has a longitudinal axis and wherein adjacent uncut tube wall regions are rotated about the longitudinal axis relative to one another.

4. The method of claim 1, wherein all of the uncut tube wall regions have the same length.

5. The method of claim 1, wherein at least some of the uncut tube wall regions have different lengths.

6. The method of claim 1, wherein all of the uncut tube wall regions have the same width.

7. The method of claim 1, wherein at least some of the uncut tube wall regions have different widths.

8. The method of claim 1, wherein the tubular member has a longitudinal axis and wherein the entire length of interconnected ribbon coil is arranged at the same angle relative to longitudinal axis.

9. The method of claim 1, wherein the tubular member has a longitudinal axis, wherein the interconnected ribbon coil includes a first section where the interconnected ribbon coil is disposed at a first acute angle relative to the longitudinal axis, and wherein the interconnected ribbon coil includes a second section where the interconnected ribbon coil is disposed at a second acute angle relative to the longitudinal axis different from the first acute angle.

10. The method of claim 1, wherein the tubular member has a proximal end, a distal end, and a length between the proximal and distal ends, and wherein the slot extends along the entire length of the tubular member.

11. The method of claim 1, wherein the tubular member has a proximal end, a distal end, and a length between the proximal and distal ends, and wherein the slot extends along only a portion of the length of the tubular member.

12. The method of claim 1, wherein the interconnected ribbon coil has a first portion having a first number of uncut tube wall regions per unit length and a second portion having a second number of uncut tube wall regions per unit length different from the first number.

13. The method of claim 1, further comprising providing a core member and disposing the interconnected ribbon coil about a portion of the core member.

14. The method of claim 13, further comprising coupling a tip member to the interconnected ribbon coil, the core member, or both.

15. The method of claim 13, further comprising coupling a shaping member to the interconnected ribbon coil, the core member, or both.

16. A medical device including the ribbon manufactured according to claim 1.

17. A medical device, comprising:
an elongate core member;
an interconnected ribbon coil disposed about a portion of the core member, the interconnected ribbon coil including a nickel-titanium alloy; and
wherein a first region of the interconnected ribbon coil has a first ribbon width and a second region of the interconnected ribbon coil has a second ribbon width different from the first ribbon width.

18. The medical device of claim 17, further comprising coupling a tip member to the interconnected ribbon coil, the core member, or both.

19. The medical device of claim 17, further comprising coupling a shaping member to the interconnected ribbon coil, the core member, or both.

20. A medical device, comprising:
an elongate core member;
a interconnected ribbon coil disposed about a portion of the core member, the interconnected ribbon coil including a nickel-titanium alloy;
wherein the interconnected ribbon coil is formed by providing a nickel-titanium alloy tubular member and laser cutting an interrupted slot in the tubular member; and
wherein a first region of the interconnected ribbon coil has a first ribbon width and a second region of the interconnected ribbon coil has a second ribbon width different from the first ribbon width.
